(19) United States
(12) Patent Application Publication
(10) Pub. No.: US 2023/0316142 A1
(43) Pub. Date: Oct. 5, 2023

(54) RADAR-BASED SLEEP MONITORING TRAINED USING NON-RADAR POLYSOMNOGRAPHY DATASETS

(71) Applicant: Google LLC, Mountain View, CA (US)

(72) Inventors: Michael Dixon, Sunnyvale, CA (US); Anupam Pathak, San Carlos, CA (US); Dongeek Shin, San Jose, CA (US); Arnaud Charton, Saratoga, CA (US); Jian Cui, Fremont, CA (US)

(73) Assignee: Google LLC, Mountain View, CA (US)

(21) Appl. No.: 18/130,731

(22) Filed: Apr. 4, 2023

Related U.S. Application Data
(60) Provisional application No. 63/327,581, filed on Apr. 5, 2022.

(51) Int. Cl. G06N 20/00

(52) U.S. Cl. CPC G06N 20/00

(57) ABSTRACT

Various arrangements are presented for training and using a machine learning model. A first training data set may be created that has more samples but fewer dimensions than a second dataset. A second set of training data, created from the second dataset, has at least one additional dimension of data than the first set of training data. An additional dimension of data can then be simulated for the first set of training data. The simulated additional dimension of data can be incorporated with the first set of training data. A first machine learning model can be trained based on the first set of training data that comprises the simulated additional dimension of data to obtain various weights. A second machine learning model can then be trained based on the second set of training data and the obtained plurality of weights from the first trained machine learning model.

### CROSS-REFERENCE TO RELATED APPLICATIONS

This application claims the benefit of, and priority to U.S. Provisional Application Ser. No. 63/327,581, filed Apr. 5, 2022, which is hereby incorporated by reference in its entirety for all purposes.

### BACKGROUND

Obtaining large datasets for training a model based on data collected by sensors can be time-consuming and expensive. Further, if the subject being monitored by the sensors are people, many people may need to be inconvenienced in order to obtain such data. For example, a polysomnography dataset may be created by conducting a clinical study in which chest straps are attached to people to monitor their sleep. Each of these people may then have their breathing measured, based on chest and abdomen movement, while asleep for multiple nights. In addition to breathing data being gathered via one or more chest straps, other vital statistic data may be gathered, such as heartrate, muscle movement, and brain activity. One or more experts trained in the science of human sleep may then manually categorize what sleep state the person is in at various times throughout the sleep session based on the sensor data and, possibly, video and audio recordings of the persons while sleeping. This process may be repeated a large number of times to create a dataset that maps gathered breathing data with a sleep state, such as awake, light sleep, REM (rapid eye movement) sleep or deep sleep, as categorized by one or more experts, for many study participants.

While such data may be useful for understanding the relationship between breathing as detected using chest movement with sleep state, the dataset cannot be immediately applied to other forms of sensors, such as those gathered by sensors other than chest straps. Conducting a new study to create such a large dataset to understand the relationship between the data output by a different type of sensor and a classification (e.g., sleep state) may be exceedingly time-consuming and cost prohibitive.

### SUMMARY

Various embodiments are described related to a method for training a machine learning model. In some embodiments, a method for training a machine learning model is described. The method may comprise creating a first set of training data from a first dataset. The first dataset may have a greater number of samples than a second dataset. The first dataset may have a fewer number of dimensions than the second dataset. Samples of the first dataset may be mapped to a plurality of ground truth states. Samples of the second dataset may have mapped to the plurality of ground truth states. The first dataset may have been gathered using a different type of sensor than the second dataset. The method may comprise creating a second set of training data from the second dataset. The second set of training data may have at least one additional dimension of data than the first set of training data. The method may comprise simulating an additional dimension of data for the first set of training data. The method may comprise incorporating the simulated additional dimension of data with the first set of training data. The method may comprise training a first machine learning model based on the first set of training data that may comprise the simulated additional dimension of data. The method may comprise obtaining a plurality of weights from the first trained machine learning model. The method may comprise training a second machine learning model based on the second set of training data and the obtained plurality of weights from the first trained machine learning model.

Embodiments of such a method may comprise one or more of the following features: the plurality of weights from the first trained machine learning model may be used as starting weights for training the second machine learning model. The first machine learning model may be a first neural network and the second machine learning model may be a second neural network. The first dataset may be a two-dimensional dataset of magnitudes of movement measurements over time. The second dataset may be a three-dimensional dataset of magnitudes of movement measurements over time and distance. Simulating the additional dimension of data for the first set of training data may comprise using noise to simulate the additional dimension of data. Simulating the additional dimension of data for the first set of training data may additionally comprise data based on the first set of training data. The method may further comprise transmitting the second machine learning model to a plurality of smart home devices. The method may further comprise classifying radar data received from a radar sensor of a smart home device of the plurality of smart home devices into a sleep state of a plurality of sleep states using the trained second machine learning model. The method may further comprise normalizing the first set of training data. The method may further comprise normalizing the second set of training data.

In some embodiments, a machine learning model system is described. The system may comprise one or more non-transitory processor-readable mediums storing a first set of training data and a second set of training data. A first dataset, used to create the first set of training data, has a greater number of samples than a second dataset that may be used to create the second set of training data. The first dataset may have a fewer number of dimensions than the second dataset. Samples of the first dataset may be mapped to a plurality of ground truth states. Samples of the second dataset may be mapped to the plurality of ground truth states. The first dataset may have been gathered using a different type of sensor than the second dataset. The second set of training data may have at least one additional dimension of data than the first set of training data. The system may comprise one or more processors. The system may comprise a memory communicatively coupled with and readable by the one or more processors and having stored therein processor-readable instructions which, when executed by the one or more processors, cause the one or more processors to simulate an additional dimension of data for the first set of training data. The one or more processors may incorporate the simulated additional dimension of data with the first set of training data. The one or more processors may train a first machine learning model based on the first set of training data that may comprise the simulated additional dimension of data. The one or more processors may obtain a plurality of weights from the first trained machine learning model. The one or more processors may train a second machine learning model based on the second set of training data and the obtained plurality of weights from the first trained machine learning model.

Embodiments of such a system may comprise one or more of the following features: the system may further comprise a plurality of smart home devices. Each of the plurality of smart home devices may comprise a radar sensor and each may be configured to use radar data received by the radar sensor of the smart home device to determine a sleep state of a plurality of sleep states using the trained second machine learning model. The plurality of weights from the first trained machine learning model may be used by the processor-readable instructions as starting weights for training the second machine learning model. The first machine learning model may be a first neural network and the second machine learning model may be a second neural network. The first dataset may be a two-dimensional dataset of magnitudes of movement measurements over time. The second dataset may be a three-dimensional dataset of magnitudes of movement measurements over time and distance. The processor-readable instructions that, when executed, cause the one or more processors to simulate the additional dimension of data for the first set of training data may comprise the processor-readable instructions, when executed, causing the one or more processors to use noise to simulate the additional dimension of data. The processor-readable instructions, when executed, may further cause the one or more processors to normalize the first set of training data and the second set of training data.

In some embodiments, a non-transitory processor-readable medium is described. The medium may comprise processor-readable instructions configured to cause one or more processors to access a first set of training data from a first dataset. The first dataset may have a greater number of samples than a second dataset. The first dataset may have a fewer number of dimensions than the second dataset. Samples of the first dataset may be mapped to a plurality of ground truth states. Samples of the second dataset may be mapped to the plurality of ground truth states. The first dataset may have been gathered using a different type of sensor than the second dataset. The one or more processors may access a second set of training data from the second dataset. The second set of training data may have at least one additional dimension of data than the first set of training data. The one or more processors may simulate an additional dimension of data for the first set of training data. The one or more processors may incorporate the simulated additional dimension of data with the first set of training data. The one or more processors may train a first machine learning model based on the first set of training data that may comprise the simulated additional dimension of data. The one or more processors may obtain a plurality of weights from the first trained machine learning model. The one or more processors may train a second machine learning model based on the second set of training data and the obtained plurality of weights from the first trained machine learning model.

Embodiments of such a medium may comprise one or more of the following: the processor-readable instructions may be further configured to cause the one or more processors to cause the second machine learning model to be transmitted to a plurality of smart home device that may be geographically distributed. The plurality of smart home devices each may use radar data captured using a radar sensor to determine a sleep state of a user using the trained second machine learning model.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an embodiment of a system for training and applying a machine learning model for sleep monitoring.

FIG. 2 illustrates an embodiment of a sleep state timeline that is mapped to sensor data.

FIG. 3 illustrates a first embodiment of a machine learning model creation system.

FIG. 4 illustrates a second embodiment of a machine learning model creation system.

FIG. 5 illustrates an embodiment of a method for training a machine learning model using datasets created using different types of sensors and having a different number of dimensions.

FIG. 6 illustrates another embodiment of a method for training a machine learning model using datasets created using different types of sensors and having a different number of dimensions.

### DETAILED DESCRIPTION

A first dataset that has been truth-tagged may be available for use in training machine learning (ML) models. The first dataset may have been created based on sensor data obtained from at least a first type of sensor. This first dataset can map sensor data to a particular ground truth. If a second type of sensor is to be used to monitor the same attribute, but creates data having a different number of dimensions, the first dataset may be used to strengthen an ML model created using the second dataset as detailed herein.

As an example of such an arrangement, consider polysomnography. A first dataset may be created via an extensive clinical study in which a large number of participants have their sleep carefully monitored. Each participant may wear multiple sensors, including one or more chest bands that measure the expansion and contraction of the participant's chest and abdomen. Additional sensors may also have been present including: brain activity sensors, heart rate sensors, video cameras, microphones, etc. The first dataset may map sensor data from the chest bands indicative of the participant's breathing and motion with a sleep state. The sleep state may have been determined by one or more sleep experts examining the available sensor data, observations of the study participant, and making a classification. The classification may be into one of a group of sleep states, such as: light sleep, deep sleep, REM sleep, or awake.

Such a first dataset is time-consuming and expensive to collect, especially if there is a large number of study participants, such as thousands of study participants. While such data may be suited for training an ML model that uses data from one or more chest straps as an input, situations may exist where a different type of sensor is to be used to collect breathing data (based on the motion of a person's chest and/or abdomen). While the ultimate sleep state classifications may be the same (e.g., light sleep, deep sleep, REM sleep, or awake) and the condition measured may be the same (e.g., rise and fall of a person's chest and/or abdomen), there may be a different number of dimensions in the data that prevent the first dataset from being used to directly train an ML model to classify a second dataset based on the output of a different type of sensor having the different number of dimensions.

Continuing with the example, a radar sensor may be desired to be used to sense the sleep state of a user. The radar data may reveal similar data to a chest band sensor: movement of the user's chest and abdomen. However, while the chest band sensor may have two dimensions of data, movement magnitude and time, the radar data can have three dimensions: movement magnitude, time, and distance.

Embodiments detailed herein are focused on how to perform ML effectively where a dataset to be used for training of an ML model has a different number of dimensions than the sensor data that will be classified by the ML model. In some embodiments, it could also be possible to perform ML effectively where a dataset used to train the ML model has the same number of dimensions as the sensor data that will be classified by the ML model, but has at least some different dimensions. As still another variation applicable to the embodiments detailed herein, ML can be performed effectively when a dataset used to train the ML model has more dimensions than the sensor data will be classified by the ML model, but the sensor data includes at least one different dimension. In such an arrangement one or more dimensions of the dataset may be ignored.

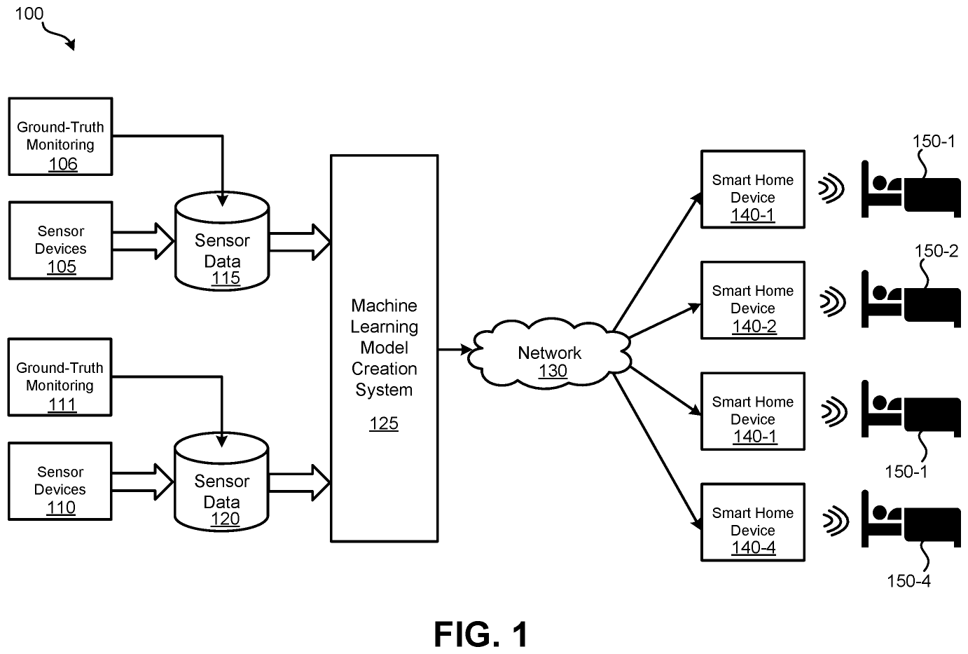

Further detail regarding such embodiments and other embodiments are provided in relation to the figures. FIG. 1 illustrates an embodiment of a system 100 for training and applying a machine learning model for sleep monitoring. System 100 can include: sensor devices 105; ground-truth monitoring 106; datastore 115; sensor devices 110; ground-truth monitoring 111; datastore 120; machine learning model creation system 125; network 130; and smart home devices 140. Datastores 115 and 120 are stored using one or more non-transitory processor-readable mediums.

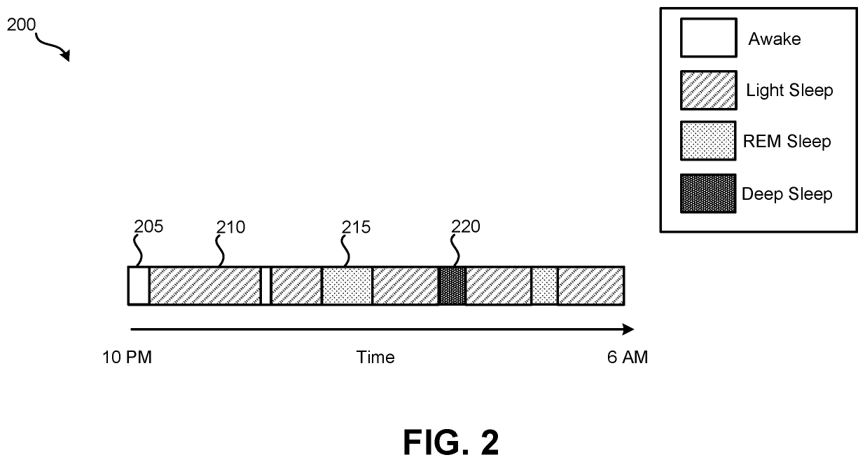

The dataset stored using datastore 115 can be sensor data that has been mapped to a ground-truth state. Sensor dataset stored using datastore 115 may be created by collecting data from sensor devices 105. Ground-truth monitoring 106 may be performed while sensor data is being collected from sensor devices 105 or may be determined after such sensor data is collected. For example, for sleep data, sensor devices 105 can include chest straps and possibly other sleep-related sensors that are used to monitor the breathing of many participants of a study. Ground-truth monitoring 106 can involve one or more sleep experts reviewing or monitoring the sleep data and assigning a particular sleep state for each time for which sensor data is received. For example, as shown in embodiment 200 of FIG. 2, if a user is monitored from 10 PM to 6 AM, a sensor datastore may store data for that particular user that indicates chest rise and fall data over time, with time ranges mapped to a particular sleep state as illustrated on timeline 201. For example, time period 205 can represent a time during which the user was classified by the one or more sleep experts as awake; time period 210 may be classified as light sleep; time period 215 as REM sleep; and time period 220 as deep sleep. The data obtained from the sensors may be mapped to the associated truth-tagged sleep state in datastore 115.

In some embodiments, the data obtained from sensor device 105 and stored in sensor dataset 115 has two dimensions: magnitude and time. For example, a sensor dataset stored using datastore 115 may be breathing data collected from a large number of clinical study participants. While sensor devices 105 and ground-truth monitoring 106 can be part of system 100, in some embodiments, this study may have been performed by a separate entity which makes datastore 115 available, such as available for download via the Internet.

Sensor datasets stored using datastore 120 can also be sensor data that is mapped to a ground-truth state. Sensor dataset stored using datastore 120 may be created by collecting data from sensor devices 110. Ground-truth monitoring 111 may be performed while sensor data is being collected from sensor devices 110 or may be determined after such sensor data is collected. For example, for sleep data, sensor devices 110 may be a different type of sensor than present in sensor devices 105. For example, a radar-based sleep monitor system may be used to monitor the breathing of some number of participants in a study. Ground-truth monitoring 111 can involve one or more sleep experts reviewing or monitoring the sleep data and assigning a particular sleep state for each time for which sensor data is received. For example, again FIG. 2 could represent the sleep states of a participant over the course of a night. The data obtained from the sensors may be mapped to the associated truth-tagged sleep state in datastore 115. While not required, generally, a sensor dataset stored using datastore 120 may be smaller, possibly significantly smaller, than sensor dataset stored using datastore 115. For example, while a sensor dataset stored using datastore 115 may have data for thousands of participants, a sensor dataset stored using datastore 120 may only have data for several hundred or even less than a hundred participants.

Sensor datasets stored using datastore 120 can have a different number of dimensions than sensor datasets stored using datastore 115. For example, while both datasets include data indicative of chest and/or abdomen movement and the same truth-tagged sleep state classifications, one of the datasets may have more dimensions. For example, sensor dataset stored using datastore 115 may have two dimensions of sensor data while sensor dataset 120 has three dimensions of sensor data. Therefore, while the sensors measured the same characteristic and were mapped to the same set of classifications, the datasets themselves have a different number of dimensions.

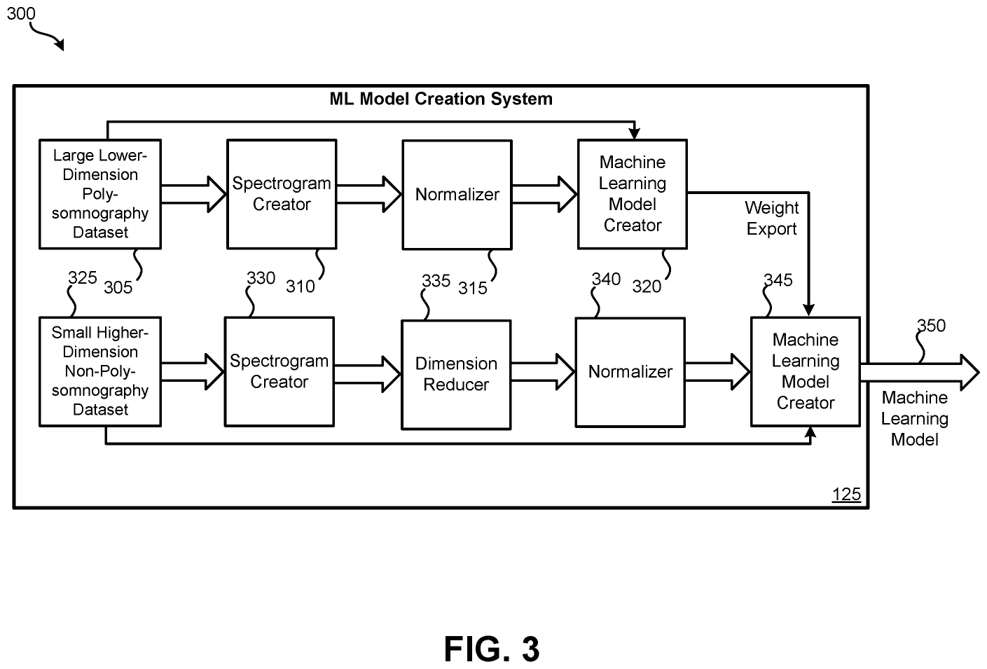

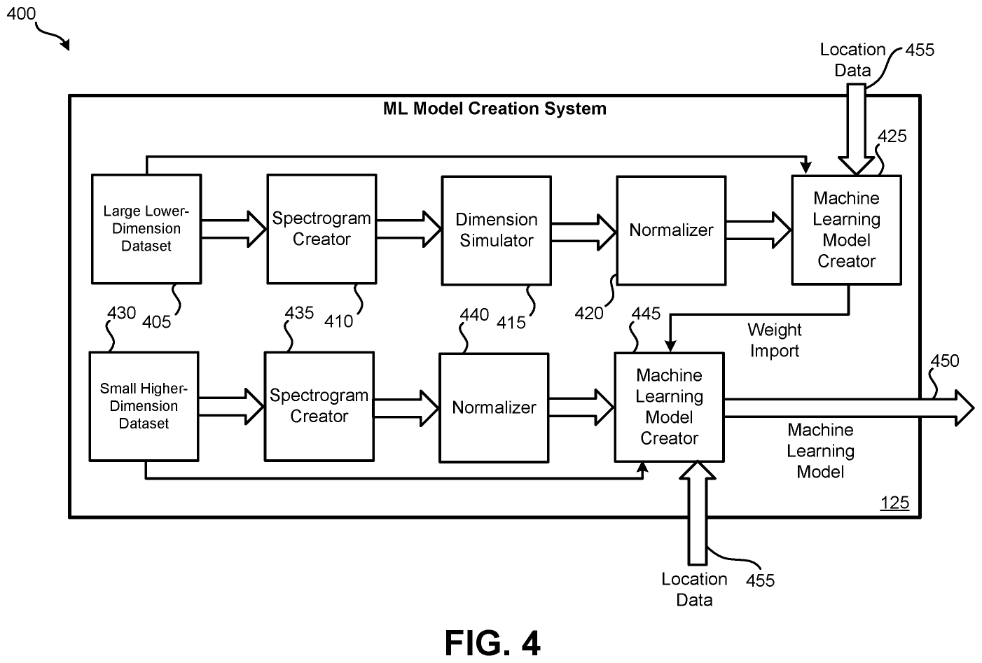

Machine learning model creation system 125, which can include one or more server systems, uses a sensor dataset stored using datastore 115 and a sensor dataset stored using datastore 120 to create a machine learning model that can be used in a similar arrangement to at least some of sensor devices 110. Machine learning model creation system 125 may include one or more special-purpose or general-purpose processors. Such special-purpose processors may include processors that are specifically designed to perform the functions of the components detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random-access memory (RAM), flash memory, a hard disk drive (HDD), or a solid-state drive (SSD). Further detail regarding the creation of a machine learning model using a sensor dataset stored using datastore 115 and sensor dataset stored using datastore 120 is provided in relation to FIGS. 3 and 4.

A machine learning model created by machine learning model creation system 125 may be installed on various devices, such as smart home devices 140, as part of a manufacturing process. Additionally or alternatively, the machine learning model can be transmitted to smart home devices 140 located at disparate locations via network 130. Network 130 can include one or more public and/or private networks, such as the Internet and a home wireless local area network. As an example, the machine learning model may be used to detect sleep state based on radar sensor data.

While the concepts of adjusting the dimensionality of datasets used to create machine learning models is applicable to many circumstances, the arrangement of FIG. 1 refers to the example of bedside smart home devices that are used to classify a user's sleep state. Smart home devices 140 may be installed bedside and can use radar to measure movement of users 150. For example, smart home devices 140 can be home assistant devices, smart home hub devices, a dedicated sleep tracking device, or some other form of smart device. For example, such functionality may be installed on a smart phone or smart watch that has a radar sensor. By performing measurements over multiple distance ranges, smart home devices 140 can detect users 150 when they move towards or away from the smart home device performing the monitoring and can distinguish from other moving objects nearby (e.g., a pet, another person).

In order to perform sleep monitoring, each of users 150 consents to being monitored by the smart home device and is provided information detailing how their sleep data is stored and used. Further, sleep monitoring may be disabled on smart home devices 140 if not desired by the respective user of users 150.

FIG. 3 illustrates an embodiment 300 of machine learning model creation system 125. For example, embodiment 300 can be used as part of system 100 or as part of another form of system that uses two datasets of different dimensions to create a trained machine learning model. While the example of embodiment 300 refers to sleep-related sensor data, it should be understood that other types of sensor data can be used instead. In embodiment 300, large lower-dimension polysomnography dataset 305 represents the dataset from datastore 115; small higher-dimension non-polysomnography dataset 325 represents the dataset from datastore 120.

Dataset 305 can be larger than dataset 325, meaning there are more samples present in dataset 305. For example, dataset 305 may have been created using a larger number of participants than dataset 325. Dataset 305 and dataset 325 are mapped to the same set of classifications, such as sleep states. These classifications were selected for datasets 305 and 325 by experts and can be assumed to be accurate. In this embodiment, the sleep states match those of FIG. 2. Dataset 305 can be a polysomnography dataset with at least some of the data corresponding to chest/abdomen movement, which is indicative of breathing. Other data present in dataset 305 can comprise brainwave activity, heartrate, video, audio, and/or muscle movement data. Processing by ML model creation system 125 of dataset 305 other than chest/abdomen movement, which may have been measured using one or more chest straps, may be ignored. Dataset 325 can also measure chest/abdomen movement but can have a different number of dimensions. For example, the chest/abdomen movement data from dataset 305 may have two dimensions: magnitude of movement and time; dataset 325 may have three dimensions: magnitude of movement, time, and distance. For example, dataset 325 may have been created based on radar sensor data that indicates movement at various distances. While dataset 305 and dataset 325 used different types of sensors to acquire data, both datasets are indicative of chest/abdomen movement (or, more generally, breathing) and are both mapped to the same classifications.

Using the chest/abdomen movement data and time data from dataset 305, spectrogram creator 310 can create a spectrogram representative of the data for each sleep session of a study participant. Each spectrogram may be created by taking a time window, such as a fixed time window size of between 5 and 30 seconds, and computing a fast Fourier transform (FFT) for that fixed time window. Such time windows may be discrete or overlapping. A magnitude may then be computed, such as a logarithmic magnitude measurement of the magnitude of the FFT. The outcome of the spectrogram creation is that the data chest/abdomen movement data from dataset 305 is converted to the frequency domain for each time window. Therefore, spectrogram creator 310 outputs a spectrogram that indicates magnitude at frequencies over time. For dataset 305, it can be expected that when a person is lying still, a peak will be present at the frequency at which the person is breathing, along with some harmonics due to breathing not being a perfectly sinusoidal event and some level of noise. When the person is moving in addition to breathing (e.g., rolling over in bed, sitting up), the spectrogram is more chaotic with a significant amount of energy scattered across many frequencies. Following spectrogram creator 310 performing processing, for each sleep session of each study participant who has data present in dataset 305, a spectrogram has been created and is mapped to the sleep state classifications set by the experts for the corresponding time.

Normalizer 315 can serve to normalize the magnitude of the spectrogram data output by spectrogram creator 310. By having a common range of magnitude (e.g., 0 to 1) with the output of normalizer 340, the magnitude ranges of frequency data will be similar. The output of normalizer 315 can then be fed to machine learning model creator 320.

The output of normalizer 315 can be fed to machine learning model creator 320. Therefore, the input to machine learning model creator 320 can be a spectrogram indicative of frequency and magnitude over time. Machine learning model creator 320 may train a machine learning model, such as a neural-network-based machine learning model that uses the normalized spectrogram and the expert-set sleep state classifications as its inputs. The trained neural network has at least an input layer, a hidden layer, and an output layer. The “synapses” that connect the “neurons” of the neural network are assigned weights as part of the training process. Once the iterative process of determining weights that can perform accurate classifications is performed, the weights may be exported from machine learning model creator 320 and imported to machine learning model creator 345. While weights may be used by neural network-based ML models, one or more other types of parameters may be used by other forms of ML models. Such other parameters may also be exported.

The neural network trained by machine learning model creator 320 can incorporate delay such that a classification for a given time period relies on normalized spectrogram data for a period of time before the given point in time and another period of time after the given point in time. Therefore, events that occur in the future from the point in time can be used by the machine learning model to make the classification. Classifications can be made using a rolling window of periods of time before and after the time period being classified.

Using the movement data, time data, and distance data from dataset 325 (which in this example was obtained from a radar sensor), spectrogram creator 330 can create a multi-dimensional spectrogram representative of the data. (In other embodiments, the dimensions need not be movement data, time, and distance—other dimensions are possible.) Since, in embodiment 300, dataset 325 has at least one additional dimension of distance, which is the distance from the radar sensor to the object (e.g., user's chest) that moved, spectrogram creator 330 functions differently than spectrogram creator 310. Similar to spectrogram creator 310, data for a fixed time window of between 5 and 30 seconds (which may match the fixed time window used by spectrogram creator 310) may be created from dataset 325. The radar sensor used to produce dataset 325 emits a chirp that is reflected back. Frequency-modulated continuous wave (FMCW) radar can be used in some embodiments; therefore, the difference in frequency between the portion of the chirp being emitted and the portion of the chirp that has been reflected and received by the radar sensor is indicative of distance.

In the example of dataset 325 being created based on data obtained from an FMCW radar sensor, for each chirp, a FFT can be performed by spectrogram creator 330. This FFT may be summed with the FFTs of the other chirps from within the time window. In FMCW radar implementations, the frequencies identified via FFT for the chirps is indicative of the distance at which a reflection occurred. Additionally, FFTs may be performed using data from multiple chirps with the same sample index (that is, received radar reflections sampled at the same time in relation to when the chirp started being emitted). For example, for each emitted chirp, 100 samples may be made of the reflections of the chirp over time, each sample being assigned a consecutively greater sample index. For example, if 30 chirps are emitted, an FFT may be performed on all samples from the reflections of those 30 chirps having a sample index of 1, a separate FFT for the sample index of 2, and so on. An FFT performed for a particular sample index across multiple chirps can measure the frequency of phase jitter across the multiple chirps, which is indicative of movement. Therefore, while the FFT of the first dimension can provide the distance at which movement is present, the FFT of the second dimension indicates a frequency of the movement.

The FFT data for ranges of sample indexes can be combined such that ranges of distance or range bins are created. For example, it may be beneficial for processing to look at radar data in 5 cm chunks of distance. The outcome of performing FFTs for individual chirps and FFTs for the same sample index across multiple chirps is that a spectrogram image is output that has the additional dimension of distance compared to the spectrographs created by spectrogram creator 410.

At this point, the spectrograms output by spectrogram creator 310 has one fewer dimension than the spectrograms output by spectrogram creator 330. Dimension reducer 335 may serve to eliminate the distance dimension from the spectrograms output by spectrogram creator 330. To do this, the distance range at which the person having their sleep state monitored is determined. Data associated with other distance ranges is discarded or ignored. The correct distance range at which the person is located that is having their sleep state monitored can be based on: a predefined distance based on user input; a calibration process; historical data; and/or determining the distance at which a highest-magnitude frequency is present that is within a range in which human breathing is expected to fall, such as between 10 to 20 breaths per minute. Spectrogram data associated with other distance ranges are discarded or not passed on to the next system component, effectively decreasing the spectrogram to a single distance range and, thus, eliminating distance as a dimension. In other embodiments, a dimension other than distance could be eliminated.

Normalizer 340 can serve to normalize the magnitude of the spectrogram data output by dimension reducer 335. By having a common range of magnitude with the output of normalizer 315, the magnitude ranges of frequency data will be similar. The output of normalizer 340 can then be fed to machine learning model creator 345. In other embodiments, normalizer 340 may be placed before dimension reducer 335.

The input to machine learning model creator 345 can be the dimensionally reduced spectrogram indicative of frequency and magnitude over time. Machine learning model creator 345 may train a machine learning model, such as a neural-network-based machine learning model that uses the normalized spectrogram and ground-truth classifications from dataset 325 as its inputs. The trained neural network has at least an input layer, a hidden layer, and an output layer. The “synapses” that connect the “neurons” of the neural network are assigned particular weights as part of the training process. As a starting point for training the neural network, rather than starting the iterative process of creating the neural network from fixed or random weights, starting points for weights may be imported from machine learning model creator 320, which allows training of a machine learning model that can perform classifications accurately using the same type of sensor data as dataset 325 even though dataset 325 is relatively small.

If an ML model was created using only dataset 325, due to the relatively small number of samples in dataset 325, the ML model would not be expected to be sufficiently accurate to perform sleep classifications in real-world scenarios. However, by starting the training of the ML model by machine learning model creator 345 with weights employed by the neural network created by machine learning model creator 320, a more accurate ML model can be created by machine learning model creator 345 using information from the smaller dataset 325, thus leveraging both dataset 305 and dataset 325, despite the different number of dimensions present in the datasets.

The output machine learning model 350 may then be distributed to various devices, such as smart home devices 140, for use in performing classifications of users' sleep. While the embodiment of FIG. 3 is focused on dataset 325 having the greater number of dimensions, dimension reducer 335 can be applied to dataset 305 if dataset 305 has the greater number of dimensions.

In other embodiments, if a first dataset has one dimension fewer than a second dataset, rather than reducing a dimension of the first dataset, an additional dimension may be added to the second dataset. FIG. 4 illustrates an embodiment 400 of machine learning model creation system 125. In embodiment 400, dataset 405 can represent the same or a similar dataset to dataset 305; dataset 430 can represent the same or a similar dataset to dataset 325. Therefore, dataset 405 can have fewer dimensions, but may have a greater number of samples. Both datasets 405 and 430 are truth-tagged with the same set of classifications. Dataset 405 and dataset 430 capture a same phenomenon but were collected using different types of sensors. For example, both datasets may capture persons' breathing patterns based on chest/abdomen movement, but use different types of sensors, such as a chest strap sensor and a radar sensor.

Using dataset 405, spectrogram creator 410 can create a spectrogram representative of the data. The spectrogram may be created by taking a time window, such as a fixed time window size of between 5 and 30 seconds, and computing a fast Fourier transform (FFT) for that fixed time window. Such time windows may be discrete or overlapping. A magnitude may then be computed, such as a logarithmic magnitude measurement of the magnitude of the FFT. The outcome of the spectrogram creation is that data (e.g., chest strap data over time) from dataset 405 is converted to the frequency domain for each time window. Therefore, spectrogram creator 410 outputs a spectrogram that indicates magnitude at frequencies over time. For dataset 405, it can be expected that when a person is lying still, a peak will be present at the frequency at which the person is breathing, along with some harmonics due to breathing not being a perfectly sinusoidal event. When the person is moving, the spectrogram is chaotic with energy scattered across many frequencies. Following spectrogram creator 410 performing processing, for each person who has data present in dataset 405, a spectrogram has been created and is mapped to the sleep state classifications of the person.

Spectrogram creator 435 can create a multi-dimensional spectrogram representative of data from dataset 430. Since dataset 430 has at least one additional dimension, which may be the distance from the radar sensor to the object (e.g., user's chest/abdomen) that moved, spectrogram creator 435 functions differently than spectrogram creator 410. Similar to spectrogram creator 410, data for a fixed time window of between 5 and 30 seconds (which may match the fixed time window used by spectrogram creator 410) may be created from dataset 430. For example, a radar sensor may be used to produce dataset 430, which may emit a chirp that is reflected back to the radar sensor.

If FMCW radar sensor data was used to create dataset 430 as detailed in relation to dataset 325, FFTs may be performed in multiple dimensions as detailed in relation to spectrogram creator 330. In embodiments that do not use FMCW radar, multiple dimensions of data may still be present that results in an additional dimension of data being present as compared with the spectrograms created by spectrogram creator 310.

The spectrograms created by spectrogram creator 435 has an additional dimension, such as distance, as compared with the spectrograms created by spectrogram creator 410. In embodiment 400, an additional dimension is added to the spectrograms created by spectrogram creator 410. This additional dimension, which in some embodiments can correspond to radar measurements at distances closer to a radar sensor than the user and farther from the radar sensor than the user, can be simulated by dimension simulator 415. Dimension simulator 415 may add noise to the dimension being added. The noise may be at least partially random. In some embodiments, real-world data may be used to simulate data for the additional dimension. For example, the spectrogram created by spectrogram creator 410 may be repeated with decreased magnitudes in the additional dimension, possibly along with injected noise. Such an arrangement can work well for simulating the additional dimension of distance between a radar sensor and a user, since the user's breathing signal will decrease as the distance from the center of the user's chest/abdomen increases. Further, noise will be expected in the additional dimension from other sources, such as movement from sources other than the user's body.

Therefore, following dimension simulator 415 simulating and adding the additional dimension to the spectrograms created by spectrogram creator 410, the spectrograms with simulated data output by dimension simulator 415 has the same number and type of dimensions as the spectrograms output by spectrogram creator 435. Normalizers 420 and 440 can serve to normalize the magnitude of the spectrogram data output by dimension simulator 415 and spectrogram creator 435, respectively. By normalizing both sets of spectrograms, differences in magnitude due to the different types of sensors used will be decreased or eliminated. Normalization may alternatively be performed at a different point within the system, such as before the additional dimension is simulated by dimension simulator 415.

The output of normalizer 420 can be fed to machine learning model creator 425. Therefore, an input to machine learning model creator 425 can be the spectrogram with the simulated dimension. For example, the spectrograms can be indicative of frequency, magnitude, and distance (which can be the simulated dimension), over time. Machine learning model creator 425 may train a machine learning model, such as a neural-network-based machine learning model that uses the normalized spectrogram with the simulated dimension and the ground-truth classifications from dataset 405 as its inputs. In some embodiments, an additional input may be present, such as location data 455, which provides an expected or known distance range at which the user is expected to be located. The trained neural network has at least an input layer, a hidden layer, and an output layer. The “synapses” that connect the “neurons” of the neural network are assigned particular weights as part of the training process. These weights may be exported from machine learning model creator 425 and imported to machine learning model creator 445. While weights may be used by neural network-based ML models, one or more other types of parameters may be used by other forms of ML models. Such other parameters may also be exported and imported.

The input to machine learning model creator 445 can be the normalized spectrograms output by normalizer 440. For example, the spectrograms can be indicative of the same dimensions as the inputs to machine learning model creator 425. Machine learning model creator 445 may train a machine learning model, such as a neural-network-based machine learning model that uses the normalized spectrogram and ground-truth classifications from dataset 430 as its inputs. The trained neural network has at least an input layer, a hidden layer, and an output layer. As a starting point for training the neural network, rather than starting the iterative process of creating the neural network from fixed or random weights, starting points for weights may be imported from machine learning model creator 425. By having a starting point for the creation of the weights that is based on a larger dataset, the weights that will ultimately be created and used for the machine learning model output by machine learning model creator 445 can be expected to result in a more accurate ML model than if only dataset 430 was used.

If an ML model was created using only dataset 430, due to the relatively small number of samples in dataset 430, the ML model would not be expected to be sufficiently accurate to perform classifications (e.g., sleep classifications) in real-world scenarios. However, by starting the training of the ML model by machine learning model creator 445 with the weights of the neural network created by machine learning model creator 425, a more accurate ML model can be created by machine learning model creator 445 being able to leverage the training performed using dataset 405. Therefore, a large portion of the training for machine learning model creator 445 is based on dataset 405 with the simulated dimension. Adjustments to the imported weights based on the training performed based on dataset 430 can be understood as “fine-tuning” adjustments that help account for particularities in the differences between the types of sensors used to create dataset 405 and dataset 430 (e.g., chest straps and radar sensors, respectively).

The output of machine learning model 450 may then be distributed to various devices, such as smart home devices 140, for use in performing classifications of users' sleep. While the embodiment of FIG. 4 is focused on dataset 430 having the greater number of dimensions, dimension simulator 415 can be applied to dataset 430 if dataset 405 has the greater number of dimensions.

While the above examples have focused on using datasets that include chest strap data indicative of breathing to train a machine learning model that will ultimately rely on radar sensor data, there are many other uses for the arrangements detailed herein. For example, rather than training a machine learning model for using radar sensor data, a machine learning model may be trained to use ultrasound data. For example, a mobile device may have an ultrasound sensor (e.g., a dedicated ultrasound sensor or a speaker and microphone that are functional at ultrasonic frequencies) that is to be used to measure breathing or some other vital statistic, such as heart rate (e.g., beats per minute). As other examples, arrangements as detailed herein can be used to help train a machine learning model that will use photoplethysmography (PPG) data, electrocardiogram (EKG), and/or accelerometer data to determine a classification, such as sleep state, based on data gathered from another type of sensor (e.g., a chest band).

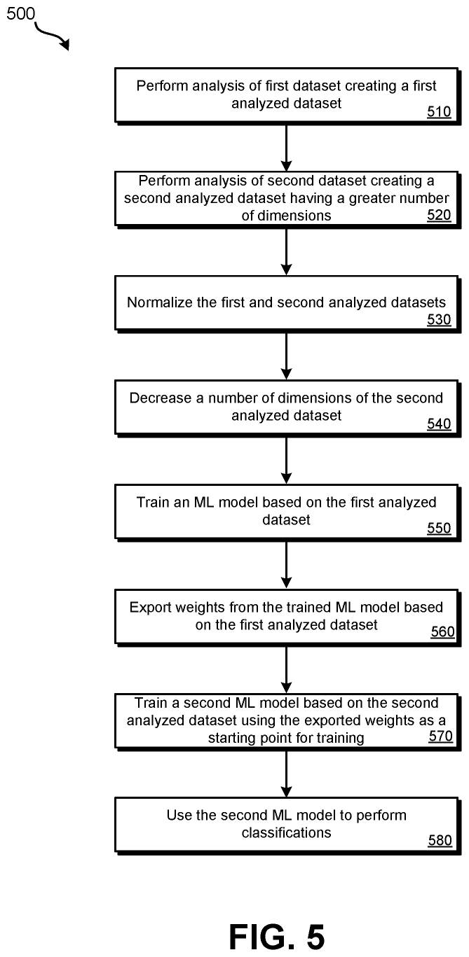

Various methods may be performed using the systems, devices, and arrangements detailed in relation to FIGS. 1-4. FIG. 5 illustrates an embodiment of a method for training a machine learning model using datasets created using different types of sensors and having a different number of dimensions. Method 500 may be performed by system 100 and specifically the ML model creation may be performed by embodiment 300 of ML model creation system 125, which can be implemented using one or more computer systems.

At block 510, a first dataset is analyzed. Comparing the first dataset to the second dataset, the following may be true: 1) the first dataset has a larger number of samples than the second dataset; 2) the first dataset and the second dataset are mapped to a same set of ground-truths, such as ground-truth values or ground-truth classifications; 3) the first dataset and the second dataset correspond to a same phenomenon, such as a person's breathing; 4) the first dataset and the second dataset were gathered using different types of sensors or devices; and 5) a different number of dimensions may be present between the first dataset and the second dataset. As detailed in relation to FIG. 3, a possible example is breathing data, where the larger first dataset is created using chest strap measurements and the smaller second dataset is created using radar sensor measurements, with both datasets being mapped to the same group of truth-tagged classifications. For this example, it is assumed that the second dataset has the greater number of dimensions, such as the second dataset having one extra dimension, which could be distance. In some embodiments, the analysis or processing of block 510 may be creating spectrograms, as detailed in relation to FIG. 3. In other embodiments, the type of processing needing to be performed may differ from, for example, applying FFTs to create spectrograms. In some embodiments, initial analysis or processing may not be needed on either dataset.

At block 520, analysis or processing of the second dataset may be performed. Due to the number of dimensions being different between datasets, the analysis or processing may differ between datasets. In this example, FFTs may be performed in two dimensions to create spectrograms that incorporate the distance dimension.

At block 530, normalization may be performed on each analyzed or processed dataset such that the magnitudes in each dataset are similar for similar classifications. For example, a user breathing while otherwise lying still in bed would result in spectrograms having approximately similar peak magnitudes. In other embodiments, normalization may be performed at a different stage in the process.

At block 540, the number of dimensions may be decreased for the second analyzed and normalized dataset. Only data present at the distance range at which the person's breathing is present may be maintained; data present at other distances may be ignored or discarded. The correct distance range at which the person is located that is having their sleep state monitored can be based on: a predefined distance based on user input; a calibration process; historical data; and/or determining the distance at which a highest-magnitude frequency is present that is within a range in which human breathing is expected to fall, such as between 10 to 20 breaths per minute.

At block 550, an ML model may be trained based on the first analyzed and normalized dataset. Therefore, for example, the ML model is trained using the created spectrograms and truth-tagged classifications for the first dataset. If a neural network ML model is created, various weights may be determined as part of the training process that are used internally by the neural network ML model. At block 560, these weights may be exported from the trained ML model based on the first dataset and using at block 570 as part of the training process for the second ML model.

At block 570, a second ML model is trained. The training of the second ML model uses the same inputs and has the same classification output as the trained ML model from block 550. Training of the second ML model uses the weights from block 560 as a starting point. The second ML model is then trained using the second dataset which has had a dimension removed. The training involving the second dataset effectively serves as fine tuning to the weights exported at block 560.

At block 580, the second ML model can then be used to perform ML classifications, such as at smart home devices. As an example, a user's sleep state can be classified as detailed in relation to FIGS. 1 through 4. While this embodiment of method 500 is focused on the second data set having more dimensions than the first dataset, the concept of eliminating one or more dimensions can be applied to the reverse situation also.

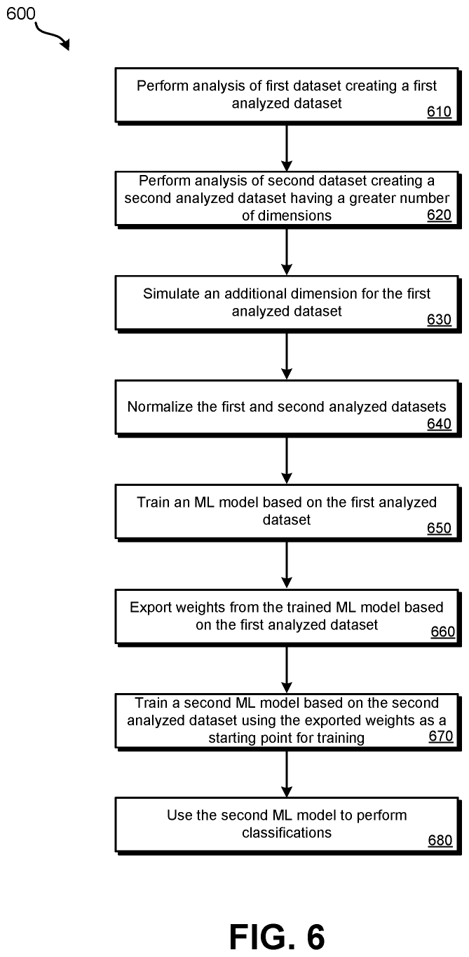

FIG. 6 illustrates another embodiment of a method for training a machine learning model using datasets created using different types of sensors and having a different number of dimensions. Method 600 may be performed by system 100 and specifically the ML model creation may be performed by embodiment 400 of ML model creation system 125 of FIG. 4, which can be implemented using one or more computer systems.

At block 610, a first dataset is analyzed. Comparing the first dataset to the second dataset, the following may be true: 1) the first dataset has a larger number of samples than the second dataset; 2) the first dataset and the second dataset are mapped to a same set of ground-truths, such as ground-truth values or ground-truth classifications; 3) the first dataset and the second dataset correspond to a same phenomenon, such as a person's breathing; 4) the first dataset and the second dataset were gathered using different types of sensors or devices; and 5) a different number of dimensions may be present between the first dataset and the second dataset. As detailed in relation to FIG. 4, a possible example is breathing data, where the larger first dataset is created using chest strap measurements and the smaller second dataset is created using radar sensor measurements, with both datasets being mapped to the same group of truth-tagged classifications. For this example, it is assumed that the second dataset has the greater number of dimensions, such as the second dataset having one extra dimension, which could be distance. In some embodiments, the analysis or processing of block 610 may be creating spectrograms, as detailed in relation to FIG. 4. In other embodiments, the type of processing needing to be performed may differ from, for example, applying FFTs to create spectrograms. In some embodiments, initial analysis or processing may not be needed on either dataset.

At block 620, analysis or processing of the second dataset may be performed. Due to the number of dimensions being different between datasets, the analysis or processing may differ between datasets. In this example, FFTs may be performed in two dimensions to create spectrograms that incorporate the distance dimension.

At block 630, an additional dimension may be added to the analyzed data of the first dataset such that the first dataset will have the same number of dimensions as the second dataset. The simulation of the additional dimension can involve adding noise and simulating signal data, such as copying the signal data from the analyzed dataset and decreasing the magnitude.

At block 640, normalization may be performed on each dataset such that the magnitudes in each dataset are similar for similar classifications. For example, a user breathing while otherwise lying still in bed would result in spectrograms having approximately similar peak magnitudes. In other embodiments, normalization may be performed at a different stage in the process.

At block 650, an ML model may be trained based on the first analyzed dataset that has had a simulated dimension added. Therefore, for example, the ML model is trained using the created spectrograms and truth-tagged classifications for the first dataset. If a neural network ML model is created, various weights may be determined as part of the training process that are used internally by the neural network ML model. At block 660, these weights may be exported from the trained ML model based on the first dataset and by using, at block 670, as a part of the training process for the second ML model.

At block 670, a second ML model is trained. The training of the second ML model uses the same inputs and has the same classification output as the trained ML model from block 650. Training of the second ML model uses the weights from block 660 as a starting point. The second ML model is then trained using the second dataset which has had a dimension removed. The training involving the second dataset effectively serves as fine tuning to the weights exported at block 660.

At block 680, the second ML model can then be used to perform ML classifications, such as at smart home devices. As an example, a user's sleep state can be classified as detailed in relation to FIGS. 1 through 4. While this embodiment of method 600 is focused on the second data set having more dimensions than the first dataset, the concept of simulating one or more dimensions can be applied to the reverse situation also.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known, processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

What is claimed is:

1. A method for training a machine learning model, the method comprising:
creating a first set of training data from a first dataset, wherein:
the first dataset has a greater number of samples than a second dataset;
the first dataset has a fewer number of dimensions than the second dataset;
samples of the first dataset are mapped to a plurality of ground truth states;
samples of the second dataset are mapped to the plurality of ground truth states; and
the first dataset was gathered using a different type of sensor than the second dataset;
creating a second set of training data from the second dataset, wherein the second set of training data has at least one additional dimension of data than the first set of training data;
simulating an additional dimension of data for the first set of training data;
incorporating the simulated additional dimension of data with the first set of training data;
training a first machine learning model based on the first set of training data that comprises the simulated additional dimension of data;
obtaining a plurality of weights from the first trained machine learning model; and
training a second machine learning model based on the second set of training data and the obtained plurality of weights from the first trained machine learning model.

2. The method for creating the machine learning model of claim 1, wherein the plurality of weights from the first trained machine learning model are used as starting weights for training the second machine learning model.

3. The method for creating the machine learning model of claim 1, wherein the first machine learning model is a first neural network and the second machine learning model is a second neural network.

4. The method for creating the machine learning model of claim 1, wherein:
the first dataset is a two-dimensional dataset of magnitudes of movement measurements over time; and
the second dataset is a three-dimensional dataset of magnitudes of movement measurements over time and distance.

5. The method for creating the machine learning model of claim 4, further comprising:
eliminating a distance dimension of the second dataset by eliminating data associated with distance ranges other than a distance range of a person being monitored.

6. The method for creating the machine learning model of claim 1, wherein simulating the additional dimension of data for the first set of training data comprises using noise to simulate the additional dimension of data.

7. The method for creating the machine learning model of claim 1, wherein simulating the additional dimension of data for the first set of training data additionally comprises data based on the first set of training data.

8. The method for creating the machine learning model of claim 1, further comprising:
transmitting the second machine learning model to a plurality of smart home devices.

9. The method for creating the machine learning model of claim 8, further comprising:
classifying radar data received from a radar sensor of a smart home device of the plurality of smart home devices into a sleep state of a plurality of sleep states using the trained second machine learning model.

10. The method for creating the machine learning model of claim 1, further comprising:
normalizing the first set of training data; and
normalizing the second set of training data.

11. A machine learning model system, comprising:
one or more non-transitory processor-readable mediums store a first set of training data and a second set of training data, wherein:
a first dataset, used to create the first set of training data, has a greater number of samples than a second dataset that is used to create the second set of training data;
the first dataset has a fewer number of dimensions than the second dataset;
samples of the first dataset are mapped to a plurality of ground truth states;
samples of the second dataset are mapped to the plurality of ground truth states;
the first dataset was gathered using a different type of sensor than the second dataset; and
the second set of training data has at least one additional dimension of data than the first set of training data;
one or more processors; and
a memory communicatively coupled with and readable by the one or more processors and having stored therein processor-readable instructions which, when executed by the one or more processors, cause the one or more processors to:
simulate an additional dimension of data for the first set of training data;
incorporate the simulated additional dimension of data with the first set of training data;
train a first machine learning model based on the first set of training data that comprises the simulated additional dimension of data;
obtain a plurality of weights from the first trained machine learning model; and
train a second machine learning model based on the second set of training data and the obtained plurality of weights from the first trained machine learning model.

12. The machine learning model system of claim 11, further comprising a plurality of smart home devices, wherein each of the plurality of smart home devices comprise a radar sensor and are each configured to:
use radar data received by the radar sensor of the smart home device to determine a sleep state of a plurality of sleep states using the trained second machine learning model.

13. The machine learning model system of claim 11, wherein the plurality of weights from the first trained machine learning model are used by the processor-readable instructions as starting weights for training the second machine learning model.

14. The machine learning model system of claim 11, wherein the first machine learning model is a first neural network and the second machine learning model is a second neural network.

15. The machine learning model system of claim 11, wherein:
the first dataset is a two-dimensional dataset of magnitudes of movement measurements over time; and
the second dataset is a three-dimensional dataset of magnitudes of movement measurements over time and distance.

16. The machine learning model system of claim 15, wherein the processor-readable instructions that, when executed, cause the one or more processors to simulate the additional dimension of data for the first set of training data comprises the processor-readable instructions, when executed, causing the one or more processors to use noise to simulate the additional dimension of data.

17. The machine learning model system of claim 15, wherein the processor-readable instructions, when executed, further cause the one or more processors to normalize the first set of training data and the second set of training data.

18. A non-transitory processor-readable medium, comprising processor-readable instructions configured to cause one or more processors to:
access a first set of training data from a first dataset, wherein:
the first dataset has a greater number of samples than a second dataset;
the first dataset has a fewer number of dimensions than the second dataset;
samples of the first dataset are mapped to a plurality of ground truth states;
samples of the second dataset are mapped to the plurality of ground truth states; and
the first dataset was gathered using a different type of sensor than the second dataset;
access a second set of training data from the second dataset, wherein the second set of training data has at least one additional dimension of data than the first set of training data;
simulate an additional dimension of data for the first set of training data;
incorporate the simulated additional dimension of data with the first set of training data;
train a first machine learning model based on the first set of training data that comprises the simulated additional dimension of data;
obtain a plurality of weights from the first trained machine learning model; and
train a second machine learning model based on the second set of training data and the obtained plurality of weights from the first trained machine learning model.

19. The non-transitory processor-readable medium of claim 18, wherein the processor-readable instructions are further configured to cause the one or more processors to cause the second machine learning model to be transmitted to a plurality of smart home device that are geographically distributed.

20. The non-transitory processor-readable medium of claim 19, wherein the plurality of smart home devices each use radar data captured using a radar sensor to determine a sleep state of a user using the trained second machine learning model.
